(19) United States
(12) Patent Application Publication
(10) Pub. No.: US 2026/0216428 A1
(43) Pub. Date: Jul. 30, 2026

(54) AUTOMATED POSITIONING OF AN INFUSION CONTAINER

(71) Applicant: CAREFUSION 303, INC., San Diego, CA (US)

(72) Inventors: Mark Smedley KELEMEN, Woods Cross, UT (US); Michael K. WORKMAN, Carlsbad, CA (US); John LANGAN, Carlsbad, CA (US); Beth R. KNAPP, Poway, CA (US); Daniel M. ABAL, San Diego, CA (US)

(21) Appl. No.: 19/147,198

(22) PCT Filed: Jan. 12, 2023
(86) PCT No.: PCT/US2023/010714

§ 371 Date: Jul. 10, 2025

(51) Int. Cl. A61M 5/168

(52) U.S. Cl. CPC A61M 5/16818; A61M 2205/3334; A61M 2205/3351; A61M 2205/502

(57) ABSTRACT

A method of adjusting a position of an infusion container includes determining a current fluid pressure of a first fluid in a fluid path at or upstream of a fluid junction. When the determined current fluid pressure satisfies a predetermined threshold: display an indication of a fault condition associated with the current fluid pressure of the fluid being above the predetermined threshold. A motor operatively coupled to an adjustment device automatically changes a height of the infusion container, relative to a reference point, to an adjusted height that causes a reduction in a flow of the first fluid beyond the fluid junction, which mixes with a flow of a second fluid from the infusion container. The method includes determining an updated fluid pressure of the first fluid in the fluid path after the motor automatically changes the height of the infusion container to the adjusted height.

### TECHNICAL FIELD

This application relates generally to monitoring and adjusting intravenous (IV) container flow rates in infusion processes.

### BACKGROUND

Medical devices such as infusion devices are used to infuse medical fluids to patients. Uncontrolled flow may occur when the infusion device delivers a fluid flow that includes a sympathetic flow of a second fluid, in addition to a first fluid.

### SUMMARY

Accordingly, there is a need for devices and methods that help improve patient safety by monitoring and detecting uncontrolled flows in an infusion process, and alerting a care provider to correct the fault condition as quickly as possible.

One method to verify the flow rate and volume delivered to a patient is to measure the volume of fluid that is consumed by the infusion pump. This is done, for example, by using a system that monitors the weight of the IV container that provides the fluid to the pump. The system measures a weight of an IV container over an infusion period and determines a rate of consumption of the volume of infusion fluids in the IV container by the decrease in weight over time. The determined rate of consumption is then compared to an expected flow rate that is programmed to be delivered by the infusion pump. The system can issue an alarm to notify a caregiver when a discrepancy in the expected flow is caused by a failure in the system.

The system has the ability to detect abnormal conditions such as faults in the pumping system that cause excessive flow or unusual flow conditions. It is desirable that a fault condition can be detected very quickly to ensure that the patient is not harmed by delayed administration of critical medications.

The disclosed devices and methods can work with any type and size of IV container, and can be used to identify a deviation from the programmed flow rate. The system can monitor sympathetic flow when primary and secondary infusion containers are used.

The disclosed subject matter also relates to a method of adjusting a position of an infusion container includes determining a current fluid pressure of a first fluid in a fluid path at or upstream of a fluid junction. In accordance with the determined current fluid pressure satisfying a predetermined threshold: displaying an indication of a fault condition associated with the current fluid pressure of the fluid being above the predetermined threshold; and causing, by a processor associated with an adjustment device, a motor operatively coupled to the adjustment device to automatically change a height of the infusion container, relative to a reference point, to an adjusted height that causes a reduction in a flow of the first fluid beyond the fluid junction, which mixes with a flow of a second fluid from the infusion container; and determining an updated fluid pressure of the first fluid in the fluid path at or upstream of the fluid junction after the motor automatically changes the height of the infusion container to the adjusted height; in accordance with the determined updated fluid pressure of the first fluid not satisfying the predetermined threshold: maintaining the infusion container at the adjusted height. The method may be implemented using a system that includes one or more processors and a memory including instructions that, when executed by the one or more processors, cause the one or more processors to perform the steps of the method described herein.

Other aspects include corresponding apparatus, and computer program products for implementation of the corresponding system and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description below, in conjunction with the following drawings. Like reference numerals refer to corresponding parts throughout the figures and description.

FIG. 1 depicts an example of an institutional patient care system of a healthcare organization, according to aspects of the subject technology.

FIG. 2 depicts an example of a system for adjusting a position of an infusion container during an infusion process, according to aspects of the subject technology.

FIG. 3A shows a system for adjusting relative height differences between a secondary infusion container and a primary infusion container, according to aspects of the subject technology.

FIG. 3B depicts a system for automatically adjusting a height of one or more infusion containers, according to some aspects of the subject technology.

FIG. 3C depicts a cross-sectional view of a telescopic pole, according to some aspects of the subject technology.

FIGS. 4A-4D depict various ways of adjusting head heights, according to aspects of the subject technology.

FIG. 5 depicts an example method for adjusting a position of an infusion container during an infusion process, according to aspects of the subject technology.

FIG. 6 is a conceptual diagram illustrating an example electronic system for adjusting a position of an infusion container during an infusion process, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

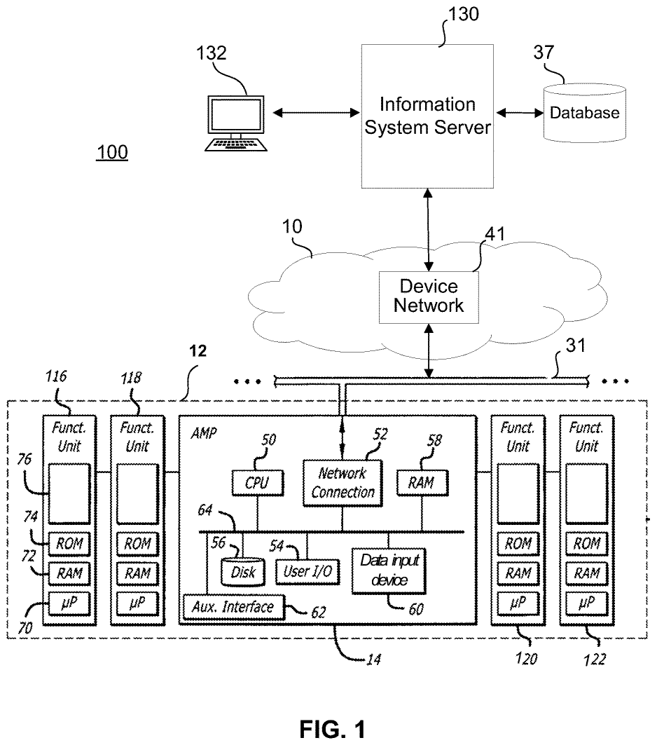

FIG. 1 depicts an example of an institutional patient care system 100 of a healthcare organization, according to aspects of the subject technology. In FIG. 1, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each patient care device 12 is connected to an internal healthcare network 10 by a transmission channel 31. Transmission channel 31 is any wired or wireless transmission channel, for example a standards-based (e.g., an 802.11) wireless local area network (LAN). In some implementations, network 10 also includes computer systems located in various departments throughout a hospital. For example, network 10 of FIG. 1 optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 41 by which patient care devices 12 (and other devices) communicate in accordance with normal operations.

Additionally, institutional patient care system 100 may incorporate a separate information system server 130, the function of which will be described in more detail below. Moreover, although the information system server 130 is shown as a separate server, the functions and programming of the information system server 130 may be incorporated into another computer, if such is desired by engineers designing the institution's information system. Institutional patient care system 100 may further include one or multiple device terminals 132 for connecting and communicating with information system server 130. Device terminals 132 may include personal computers, personal data assistances, mobile devices such as laptops, tablet computers, augmented reality devices, or smartphones, configured with software for communications with information system server 130 via network 10.

Patient care device 12 comprises a system for providing patient care, such as that described in U.S. Pat. No. 5,713,856 to Eggers et al., and U.S. Pat. No. 10,732,798 to Langan et al, which are each incorporated herein by reference for this purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a control module 14, also referred to as interface unit 14, connected to one or more functional modules 116, 118, 120, 122. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally or in the alternative, user interface device 54 could include specifically configured means for displaying and inputting information, such as a monitor, a printer, a keyboard, softkeys, a mouse, a track ball and/or a light pen. Data input device 60 may be a bar code reader capable of scanning and interpreting data printed in bar coded format. Additionally or in the alternative, data input device 60 can be a specifically configured device for entering coded data into a computer, such as a device(s) for reading a magnetic strips, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1 to be disposed within interface unit 14, it is recognized that data input device 60 may be integral within pharmacy system 34 or located externally and communicating with pharmacy system 34 through an RS-232 serial interface or other appropriate communication means configured to enable all or portions of the features described. Auxiliary interface 62 may be an RS-232 communications interface, however other means for communicating with a peripheral device such as a printer, patient monitor, infusion pump or other medical device may be used without departing from the subject technology. Additionally, data input device 60 may be a separate functional module, such as modules 116, 118, 120 and 122, and configured to communicate with controller 14, or other system on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

Functional modules 116, 118, 120, 122 are specifically configured devices for providing care to a patient or for monitoring patient condition. As shown in FIG. 1, at least one of functional modules 116, 118, 120, 122 may be an infusion pump module such as an intravenous infusion pump for delivering medication or other fluid to a patient. For the purposes of this discussion, functional module 116 is an infusion pump module. Each of functional modules 118, 120, 122 may be specifically configured patient treatment or monitoring device including, but not limited to, an infusion pump, a syringe pump, a PCA pump, an epidural pump, an enteral pump, a blood pressure monitor, a pulse oximeter, an EKG monitor, an ETCO2 capnography module, an EEG monitor, a heart rate monitor or an intracranial pressure monitor or the like. Functional module 118, 120 and/or 122 may be a printer, scanner, bar code reader or other specifically configured peripheral input, output or input/output device.

Each functional module 116, 118, 120, 122 communicates directly or indirectly with interface unit 14, with interface unit 14 providing overall monitoring and control of device 12. Functional modules 116, 118, 120, 122 may be connected physically and electronically in serial fashion to one or both ends of interface unit 14 as shown in FIG. 1, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 116, 118, 120, 122 may include module-specific components 76, a microprocessor 70, a volatile memory 72 and a nonvolatile memory 74 for storing information. It should be noted that while four functional modules are shown in FIG. 1, other numbers of devices may be connected directly or indirectly to central controller 14. The number and type of functional modules described herein are intended to be illustrative, and in no way limit the scope of the subject technology. Module-specific components 76 include components necessary for operation of a particular module, such as a pumping mechanism for infusion pump module 116.

While each functional module may be capable of a least some level of independent operation, interface unit 14 monitors and controls overall operation of device 12. For example, as will be described in more detail below, interface unit 14 provides programming instructions to the functional modules 116, 118, 120, 122 and monitors the status of each module. The programming instructions may be based a volume or flow rate detected using at least some of the features described.

Patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 10 location in the hospital or hospital computer network. Patient care information may be entered through interface device 52, 54, 60 or 62, and may originate from anywhere in network 10, such as, for example, from a pharmacy server, admissions server, laboratory server, and the like.

Medical devices incorporating aspects of the subject technology may be equipped with a Network Interface Module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction or a combination of both automated and manual interaction. Automated interaction may be continuous or intermittent and may occur through direct network connection 54 (as shown in FIG. 1), or through RS232 links, MIB systems, RF links such as BLUETOOTH, IR links, WLANS, PANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in health information server (HIS) 30, decision support 48, remote data server 49, hospital department or unit stations 46, or within patient care device 12 itself.

Direct communications with medical devices operating on a network in accordance with the subject technology may be performed through information system server 30, also known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication.

Flow rate refers to the volume of fluid that is displaced over time, and can be either instantaneous flow or cumulative flow. Instantaneous flow is the volume of flow over a fixed period of time. Cumulative flow is the cumulative value of flow from the start to the finish of the measurement. For example, a 500 mL of medication infused over a period of 8 hour, represents a cumulative flow rate of (500 mL/8 hour) or 62.5 mL/hr. The pump flow rate depends on factors such as tubing dimensions (e.g., inner diameter, length, material properties), mechanism speed (e.g., cam rotations per minute (RPM)), and tubing occlusion which is the amount of squeeze applied to the tubing to create a fluid seal. The pump flow rate is also dependent on the tubing wall thickness and occluder finger force. Nonconformance in any of these or other fault conditions can result in uncontrolled flow rate or volume delivered by the pump. Thus, the ability to verify the flow rate and volume delivered by the pump, present a notification of any aberrant condition, and, if possible, adjust one or more medical devices may help to ensure and enhance patient safety.

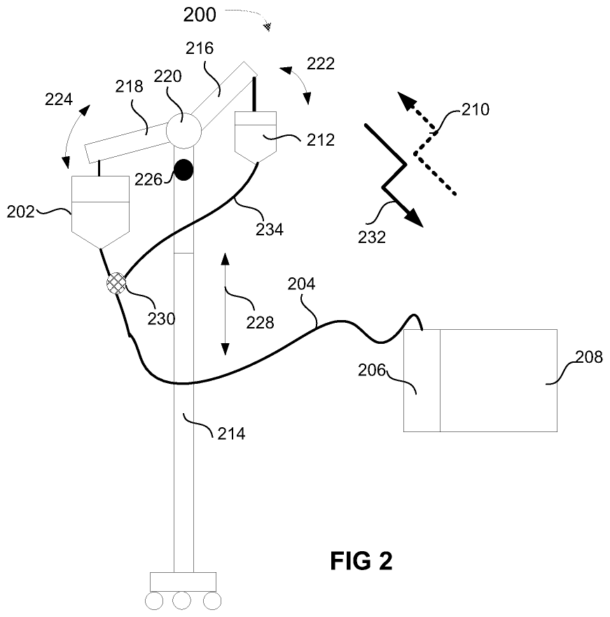

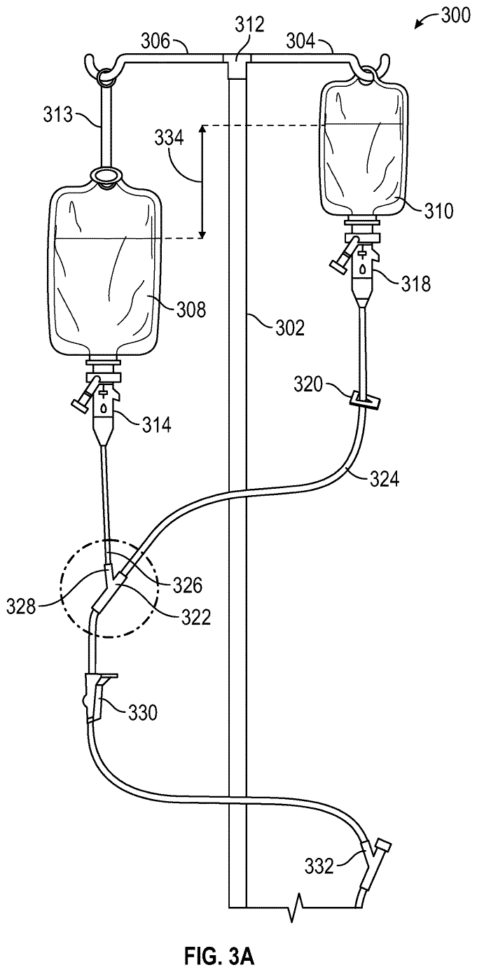

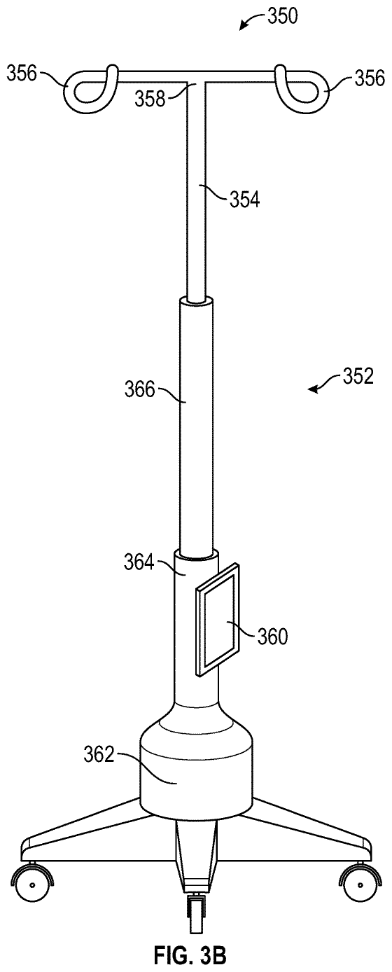

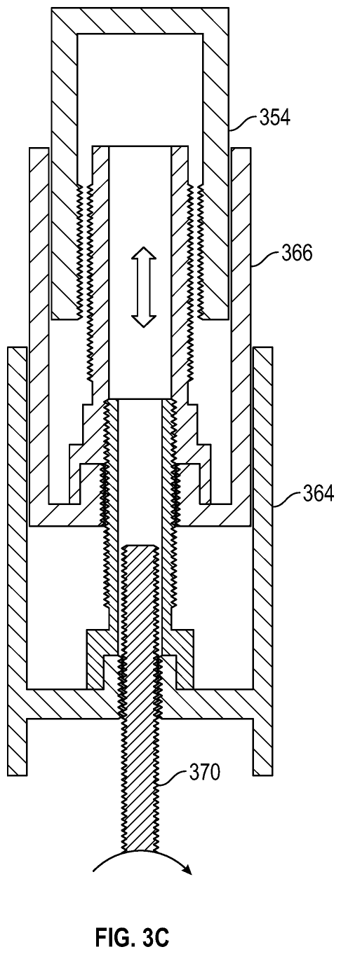

FIG. 2 depicts an example of a system for adjusting a position of an infusion container during an infusion process, according to aspects of the subject technology. A system 200 includes an adjustable pole 214, an example of which is described in FIGS. 3B and 3C. The adjustable pole 214 may extend or retract along a direction 228. The adjustable pole 214 includes a pivot 220 from which a first arm 216 and a second arm 218 are supported. An example of an adjustable pole having pivotable arms is described in FIG. 3A. A secondary infusion container 212 is supported on the first arm 216, and a primary infusion container 202 is supported on the second arm 218. The first arm 216 is pivotable about an angular range 222. Pivoting the first arm 216 changes a height (e.g., an absolute height) of a fluid level in the secondary infusion container 212. As portrayed in the depicted example, the pivot 220 may at a first proximal end of the arm such that, when the arm pivoted about the pivot 220, a second distal end is raised (or lowered).

Similarly, pivoting the second arm 218 changes a height (e.g., an absolute height) of a fluid level in the primary infusion container 202. The first arm 216 and the second arm 218 can be pivoted independently of each other, allowing a relative height difference between the primary infusion container 202 and the secondary infusion container 212 to be changed. Infusion containers are typically hung from IV poles or other fixture proximate to an infusion pump 206 at a specific head height to afford an appropriate gravitational pull on the fluid (e.g., fluid pressure) for the infusion.

In some implementations, additionally or in the alternative to pivot 220, the first arm 216 and/or the second arm 218 may include a telescoping portion. For example, an arm 216 or 218 may include two portions 216a and 216b or 218a and 218b, with one portion retracting into and/or extending from within the other portion. In this regard, instead of pivoting, a respective arm may telescope to extend in length, thereby raising the height of the corresponding container 202, 212. Conversely, the arm may retract to lower the height of the container. In some implementations, to obtain a modified height, the arm may pivot partially while extending (or retracting) in length. As the motor of pivot 220 rotates, the telescopic members may shift out or in. While the examples herein describe implementations that include pivoting arms, it should be understood that such implementations may be substituted with telescoping functionality, and/or supplemented with such functionality.

In some implementations, a sensor 226 (e.g., a camera or vision system, or a load cell) is provided within or on a portion of the adjustable pole 214. For example, a load cell measures the weight of the primary infusion container 202 that is hanging from the second arm 218. The weight is converted to fluid volume using the known density of the fluid inside the infusion container. The density may be known based on information provided to the system 200 through a user input or via a wireless message. This volume is then monitored over a period of time to determine the average flow rate that is being directed to an infusion pump 206. The infusion pump 206 is controlled by an infusion device 208, which may control additional infusion pumps in addition to the infusion pump 206. The primary infusion container 202 is connected via a tubing 204 to the infusion pump 206. In some implementations, the secondary infusion container 212 is connected via an administration set 234 to a primary port 230. The primary port is a fluid junction. In some implementations, while the secondary infusion container is being infused, the fluid from the primary infusion container 202 is stopped by a check valve (e.g., a backcheck valve) in the primary port 230 from being mixed into the fluid flow.

As used herein, a “head height” relates to a height between a patient and a fluid source that influences a fluidic pressure at which a fluid from the fluid source is delivered to the patient. In some implementations, the head height is a vertical distance between a patient (e.g., an injection site at the patient, a position of a patient's heart) and a top fluid level of a fluid in an infusion container. In some implementations, the head height is a vertical distance between a patient and a portion (e.g., a top portion, a middle portion, or a bottom portion) of an infusion container. When a head height differential (e.g., a difference between a head height of a secondary infusion container 212 and a head height of a primary infusion container 202, each of the two head heights is a vertical height difference between the respective infusion container and the patient) between the primary infusion container 202 and the secondary infusion container 212 is insufficient, the check valve may be unable to fully prevent a flow from the primary infusion container from mixing with the flow from the secondary infusion container 212. Such a flow is termed “concurrent flow” or “sympathetic flow.” In some implementations, a pressure sensor near or in the fluid junction detects a pressure spike prior to the concurrent flow occurring. A processor associated with the adjustable pole 214 calculates a change in height of the secondary infusion container 212 and/or the primary infusion container 202 to provide an appropriate head height differential that is sufficient to stop the concurrent flow. A processor in electronic communication with a motor connected to the pivot 202 is able to cause an adjustment in the heights of one or more of the first arm 216 and the second arm 218. Adjusting the head height differential helps to reduce (e.g., eliminate) the detected pressure spike.

In some implementations, the pressure measurements are made by the infusion pump 206, and relayed as signal data 210 to the adjustable pole to change a height of one or more of the first arm 216 and the second arm 218. In some implementations, the adjustable pole 214 transmits a signal 232 to the infusion pump 206 regarding detection measurements captured by the adjustable pole 214. For example, the adjustable pole 214 may transmit weight measurements to the infusion pump. The adjustable pole 214 may include a vision system that monitors for the presence of drop formation in a drip chamber associated with the primary infusion container 202 (as shown in FIG. 3A).

According to various implementations, a computing device (e.g., within the infusion device 208) may continuously monitor the fluid level within the container(s) and continuously adjust height of the container(s) to maintain a particular flow rate or pressure. For example, the computing device may continuously adjust the length of the pole 214 and/or continuously pivot or adjust the length of the arms 216, 218. As the fluid depletes, the computing device detects the new fluid level and adjusts the height. In some implementations, the computing device may monitor pressure and, on detecting a pressure spike reaching a certain threshold (e.g., dangerous threshold), the computing device may automatically adjust the pole and/or arms to adjust the height(s) of the respective container(s) until the spike is no longer detected.

FIG. 3A shows a system for adjusting relative height differences between a secondary infusion container and a primary infusion container, according to aspects of the subject technology. The system 300 includes a pole 302 having a junction 312 positioned at a top portion of the pole 302. A first arm 304, a second arm 306, and the pole 302 are connected by the junction 312. A secondary infusion container 310 is secured to (e.g., suspended from) the first arm 304 (e.g., the secondary infusion container 310 is attached to a hook of the first arm 304), and a primary infusion container 308 is secured to (e.g., suspended from) the second arm 306 (e.g., the primary infusion container 308 is attached to a hook of the second arm 306).

In some implementations, an infusion process to delivery secondary medications may involve a secondary infusion container 310 in addition to the primary infusion container 308. The primary infusion container 308 is fluidically connected to a primary administration set 326. In some implementations, the primary infusion container 308 is connected to a drip chamber 316 and then to the primary administration set 326. The secondary infusion container 310 is fluidically connected to an administrative set 324, sometimes also called a “piggy-back set.” In some implementations, the administrative set 324 is a short administrative set that delivers the fluid in the secondary infusion container 310 through the primary administration set 326. In some implementations, a drip chamber 318 is located between the secondary infusion container 310 and the administration set 324. The drip chamber 318 allows a healthcare provider to visualize the flow rate (e.g., via a drip rate) from the secondary infusion container 310.

In some implementations, an extension hook 313 is used to attach the primary infusion container 308 to the second arm 306, for example, to lower a height of the primary infusion container 308 relative to a height of the secondary infusion container 310. As shown in FIG. 3A, the difference between the heights of the secondary infusion container 310 and the primary infusion container 308 is known as a head height differential 334. The primary administration set 326 is connected to the administration set 324 of the secondary infusion container 310 by a primary port 322. The primary port 322 is a fluid junction and, in some implementations, includes a backcheck valve 328. The head height differential 334, in conjunction with the backcheck valve 328, which is normally closed to the fluid in the primary infusion container 308, ensures that little or no fluid from the primary infusion container 308 flows past the primary port 322 to a downstream secondary port 332 during infusion of the secondary infusion container 310. Instead, fluid from the secondary infusion container 310 would flow through the primary port 322 to the downstream secondary port 332. Downstream of the primary port 322 is a clamp 330 that can be used to control a flow of the infusion fluid to the secondary port 332.

A sufficiently large head height differential 334 provides enough hydrostatic pressure from the secondary infusion container 310 to prevent the backcheck valve 328, to the primary line, from opening. As the infusion of the secondary infusion container 310 progresses, a fluid height of the secondary infusion container 310 drops, leading to a reduced head-height differential 334. When the head height differential 334 becomes too small, the backcheck valve may start to open, causing fluid from the primary infusion container 308 to start flowing through the primary port 322 to the secondary port 332. A sympathetic flow occurs when fluids from the primary infusion container 308 and secondary infusion container 310 are flowing at the same time. A sympathetic flow is also called a concurrent flow. Concurrent flow can occur when a line pressure associated with (e.g., in) the secondary infusion container is not sufficient to keep the backcheck valve 328 closed. Consequently, some amount of fluid from the primary infusion container is infused, which lowers the effective infusion rate of the secondary infusion container. Thus, a sympathetic flow may cause a delay in the delivery of the secondary infusion.

The primary set may be connected to an infusion pump (e.g., a large volume peristaltic infusion pump) that directly controls a flow rate from the secondary infusion container 310 (the “secondary flow rate”). One or more of the following conditions may cause a sympathetic flow to occur (e.g., closure of the backcheck valve 328 is no longer maintained): when the head height differential 334 is too small, when the secondary flow rate is too high, when the administration set 324 is occluded or not opened fully to the primary port 322, or when air enters the backcheck valve 328 or backcheck valve component defects.

A mismatched height differential between the secondary infusion container 310 and the primary infusion container 308 can cause disruptions to the infusion process. For example, when a relative height between the primary infusion container 308 and the secondary infusion container 310 is too small, a hydrostatic pressure within the primary infusion container 308 may increase (e.g., due to the larger volume of fluid in the primary infusion container 308 vis-à-vis a decreasing volume of fluid in the secondary infusion container 310) such that a pressure spike is generated within the primary administration set 326, upstream of the backcheck valve 328, causing the backcheck valve 328 to open and a sympathetic flow of the fluid from the primary infusion container past the primary port 322 toward the secondary port 332 to occur.

In some implementations, the pressure spike is detected as a pressure difference between two ends of the backcheck valve 328. When the flow rate from the secondary infusion container 310 is high or is increased, a dynamic fluid pressure within the secondary administration set 324 at the primary port 322 is lowered. The primary port 322 may be formed to support the clinical need such as in a Y-shape or other arrangement suited to create the desired fluid path for the therapy and patient. Even when the hydrostatic pressure in the primary administration set 326 upstream of the backcheck valve 328 remains constant, a pressure spike results due to the drop in the fluidic pressure at the primary port 322. This pressure spike causes the backcheck valve 328 to open, and the fluid from the primary infusion container 308 to be mixed into the fluid flow downstream of the primary port 322.

The sympathetic flow of the fluid from the primary infusion container 308 past the primary port 322 toward the secondary port 332 may be preceded a pressure spike in the fluid path associated with the primary infusion container 308. In some implementations, determining a fluid pressure associated with the primary infusion container 308 includes inferring a fluid pressure of the fluid in the fluid path by detecting a formation of drops in the drip chamber 316 upstream of the backcheck valve 328. In some embodiments, fluid pressure is determined using a pressure sensor of an infusion pump that detects a fluid pressure upstream of the infusion pump. For example, the infusion pump has a pressure sensor that measures a joint fluid pressure of a flow from the secondary infusion container 310 and a flow from the primary infusion container 308. A processor in the infusion pump, in response to receiving input from the pressure sensor of the infusion pump that a spike in fluid pressure has been detected, determines if the pressure spike exceeds a predetermined fluid pressure threshold. In some implementations, the predetermined fluid pressure threshold indicates a high probability of a sympathetic flow occurring at pressures above the pressure threshold. In some implementations, the predetermined fluid pressure threshold is a pre-calibrated value associated with one or more of: characteristics of the backcheck valve 328 (e.g., cracking pressure associated with the backcheck valve), a volume of the primary infusion container 308, characteristics of the primary administration set 326 (e.g., a tubing diameter, or a tubing length), a volume of the secondary infusion container 310, characteristics of the secondary administration set 324 (e.g., a tubing diameter, or a tubing length), characteristics of the primary port 322 (e.g., dimensions of the primary port 322), and a model of the infusion pump.

In some implementations, determining a fluid pressure associated with the primary infusion container 308 includes detecting a fluid pressure along the primary administration set 326, or the infusion line, using a pressure sensor provided (e.g., integrated with) near (e.g., upstream of, or downstream of) the backcheck valve 328. In some implementations, the drip chamber 316 further includes a fluid pressure sensor; detecting using a pressure sensor provided near the drip chamber). In some implementations, the drip chamber 316 and the drip chamber 318 are used during a gravity infusion. In some implementations, the drip chamber 316 and the drip chamber 318 are used together with an infusion pump.

In some implementations, an infusion process may incorporate an intentional sympathetic flow at various time points within the infusion process. For example, when an intentional sympathetic flow occurs, the volume of the fluid in the primary infusion container 308 decreases, causing the fluid level in the primary infusion container 308, thereby increasing the head-height differential 334 once again (and causing the sympathetic flow to stop). The sympathetic flow may thus occur periodically, stopping when a sufficiently large head-height differential 334 is obtained.

In order to maintain an appropriate head-height differential 334 to reduce (e.g., prevent) a sympathetic flow during the infusion of the secondary infusion container 310, an adjustment device is used to control and adjust a relative height difference between arm 304 and the arm 306. In some implementations, prior to the start of an infusion, the adjustment device receives information about a volume to be infused (VTBI), a programmed rate of infusion for the secondary infusion container, and an upstream pressure. A detection algorithm may include one or more of the following factors: a programmed rate, the VTBI, a bag volume, and a line pressure to determine if a sympathetic flow would occur. A concurrent limit, which is a function of a secondary bag volume and a flow rate, is used to determine when a sympathetic flow occurs. When a detected line pressure is less than the concurrent limit, the detection algorithm would send a control signal to the processor indicating that the system detects sympathetic flow from the primary infusion container. In some implementations, an increase in a flow rate of the secondary infusion container causes the line pressure associated with the secondary infusion container to fall, allowing the backcheck valve 328 to open.

In some implementations, the upstream pressure may be a joint expected pressure generated based at least in part on the pressure from the primary infusion container and the pressure from the secondary infusion container as measured by a pressure sensor of the infusion pump. In some implementations, the upstream pressure is measured by a first pressure sensor in (or associated with) the secondary line and a second pressure sensor in (or associated with) the primary line. In some implementations, the adjustment device uses the pressure sensors, or additional sensors (e.g., a visual system to monitor the formation of drops in a drip chamber, a weight sensor to measure if a weight of the primary infusion container decreases during infusion) to determine if the backcheck valve 328 is opened during the infusion process.

The adjustment device can present instructions to a user to prompt the user to increase the height (e.g., the head height) of the secondary infusion container. In the absence of user actions, the adjustment device may engage the motor to direct the system 300 or 350 to increase the height of the secondary infusion container 310 relative to the primary infusion container 308.

The adjustment device may be integrated with the pole 302 (e.g., at a base of the pole 302, within a housing connected with the pole 302) or the adjustment device may be a separate device that is in electronic communication with the pole 302. The adjustment device is in electronic communication with a motor housed within the pole 302, the pressure sensor, and the infusion pump (e.g., for non-gravity infusion). The adjustment device induces circuitry and a processor that calculates the a head-height differential necessary to counteract a sympathetic flow, based on the detected fluid pressure measurements.

In some implementations, to reduce or eliminate sympathetic flows from the primary infusion container 308, a height of the secondary infusion container 310 is raised to increase the head-height differential 334. In addition, or alternatively, the second arm 306 can be lowered to increase the head-height differential 334.

In some implementations, in addition, or alternatively to the fluid pressure measurements, the system 300 includes a weight sensor to monitor a weight of the primary infusion container 308. The weight sensor may include circuitry and a microprocessor that calculates the fluid volume by converting the measured weight signal. In some implementations, when no fluid flow is desired form the primary infusion container, any decrease in weight detected by the weight sensor may be communicated using either a wired or wireless signal from the weight sensor to the infusion device 208 to present an alert to the user that a sympathetic flow has occurred or is occurring. Prior to the start of the infusion process, the infusion device 208 sends a signal data 210 to the weight sensor regarding the programmed infusion (e.g., and a duration of the secondary infusion process for which no decrease in weight of the primary infusion container 308 is desired.) In some implementations, the weight sensor is incorporated as part of the infusion container. Since the infusion containers are generally disposable and not reused for multiple infusions, the weight sensor and circuitry may be low cost and imbedded into the infusion container and would not be removed from the infusion container once it is used.

In some implementations, a secondary infusion container is infused first after which a primary infusion container infuses. Generally, no mixing is desired. However, differences in height, or pump pressure fluctuations can allow sympathetic flow (mixing) of the two fluids. The system may be used to continuously monitor the fluid volumes in each bag and alert the user if the mixing exceeds 5% from the primary bag. The system may also alert the user of excessive sympathetic flow or when the secondary bag is emptied. In other words, the system may also maintain a record of the infusion.

FIG. 3B shows a system for automatically adjusting a height of one or more infusion containers, in accordance with aspects of the subject technology. A system 350 includes a telescopic pole 352 having an upper extension 354 that includes one or more one or more hooks 356. A force sensor 358 is provided within or on the upper extension 354. The system 350 also includes a digital display and device interface 360 that displays pertinent information to a care provider or to the patient. For example, the display may show the desired medication and flow rate, as an additional way of confirming various parameters in the infusion process.

Included within the housing of system 350 is a motor 362, configured to telescopically actuate one or more cylindrical segments 364, 366, and 354 of the telescopic pole 352, as shown in FIG. 3C. In some implementations, the system 350 is mounted on wheels to improve portability of the system 350. The fluidic pressure available from an infusion container is proportional to a height of the infusion container (or a top fluid level of a fluid within the infusion container) above an injection site, or the “head height”. By increasing the height of the pole 352, an infusion container secured to a hook in the upper extension 354 can provide increased infusion pressure. An increased infusion pressure may change a flow rate of the fluid from the infusion container to the patient. The system 350 also includes a processor configured to receive information about an infusion container size and volume. The force sensor 358 is used to monitor a mass of the infusion container. In some implementations, a height of the infusion container is adjusted throughout the infusion to maintain the desired head height.

In some implementations, a medical care provider selects or inputs the infusate and the desired flow rate. The processor in system 350 computes a height change Δh, for the flow rate Q to be achieved using the following Bernoulli's equation for flow rate:

$$Q = π {r}^{2} \sqrt{2 g ( Δ h + \frac{Δ P}{ρ g} + \frac{{V}_{0}^{2}}{2 g} - ρ {gf}_{n} )}$$

where πr² is a cross-sectional area of the tubing in the administration set, ΔP is the pressure gradient, V₀ is the initial velocity of the fluid flow, and ƒₙ is a friction factor.

FIG. 3C shows a cross-sectional view of a telescopic pole, according to some aspects of the subject technology. FIG. 3C shows three different cylindrical segments 364, 366, and 354 of the telescopic pole 352. The cylindrical segment 364 is an outermost cylindrical portion having a largest diameter and at least partially and concentrically surrounding the cylindrical segment 366, which is an intermediate cylindrical segment having a diameter smaller that the cylindrical segment 364. The cylindrical segment 366 at least partially and concentrically surrounds the cylindrical segment 354, which is an innermost cylindrical segment having a second cylindrical segment 366 smaller diameter compared to the first cylindrical segment 364 and an innermost cylindrical segment 354 each of the three cylindrical portions are connected by a central threaded supporting beam that can be rotated in a clockwise direction to cause a separation between different segments cylindrical portions to increase the height of the pole 352. By increasing a separation between one or more cylindrical segments 364, 366, and 354 of the telescopic pole 352, for example, by rotating a threaded component 370 supporting the three cylindrical segments 364, 366, and 354 in a first rotation direction (e.g., clockwise rotation), a height of the telescopic pole 352 is increased. The threaded component 370 may be rotated by a rotary motor. Conversely, by decreasing a separation between one or more cylindrical segments 364, 366, and 354 of the telescopic pole 352, for example, by rotating the threaded component 370 supporting the three cylindrical segments 364, 366, and 354 in a second rotation direction (e.g., counterclockwise rotation), a height of the telescopic pole 352 is decreased.

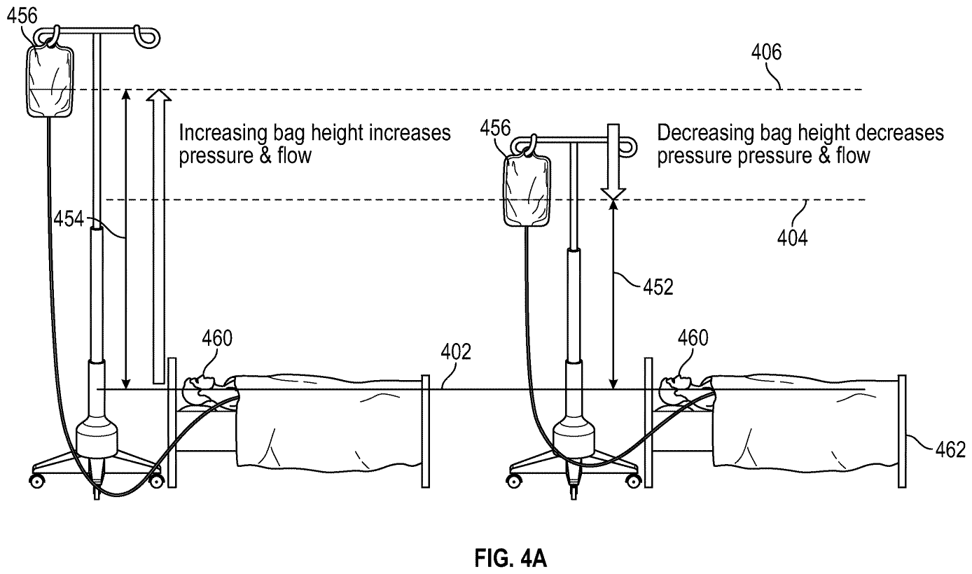

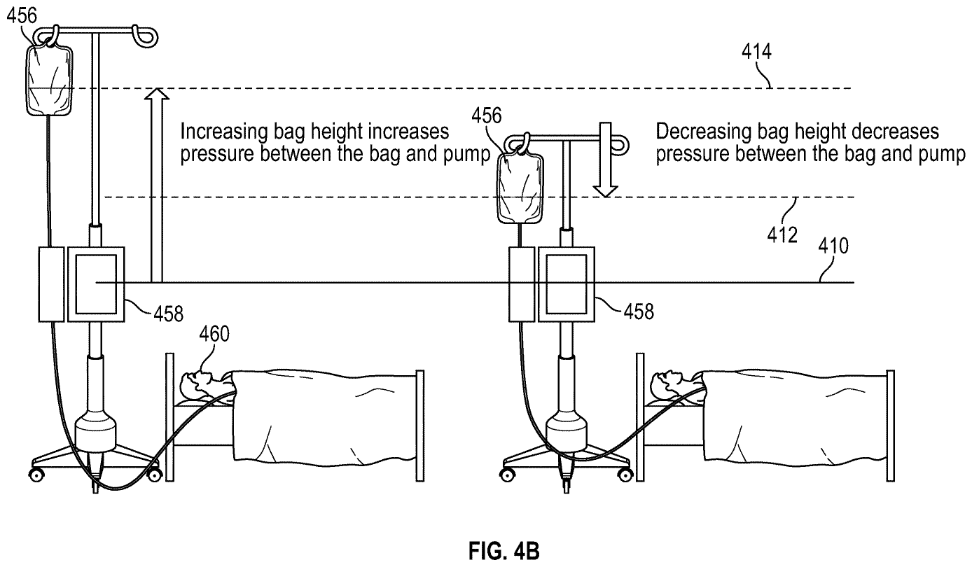

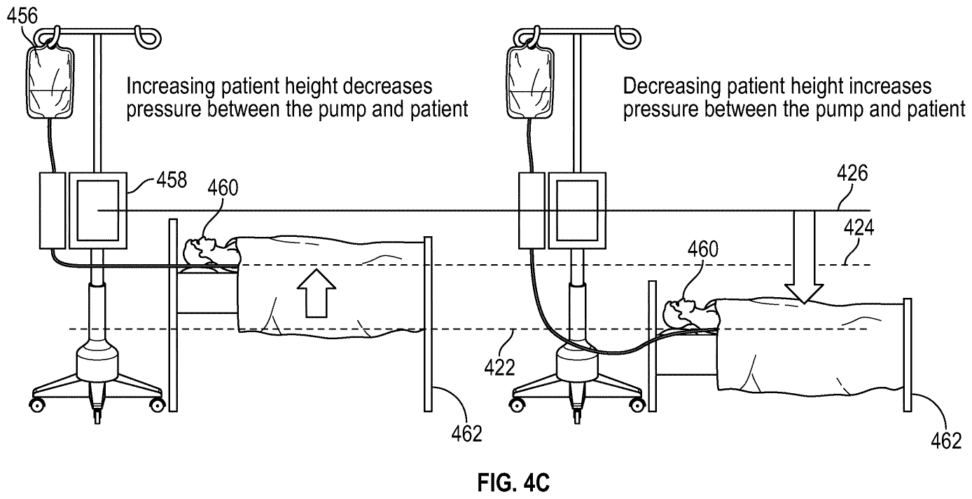

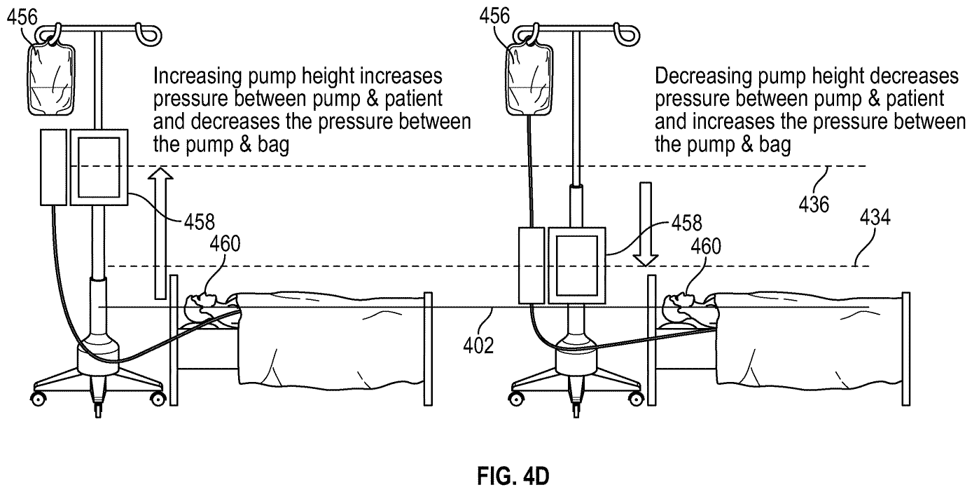

FIGS. 4A to 4D show various ways of adjusting head heights, according to aspects of the subject invention. FIG. 4A shows a patient 460 lying at a height 402. Instead of a patient lying horizontally (e.g., in a hospital bed), the patient 460 may also be sitting or standing, and the height 402 may then be associated to a height of where the IV administration set is inserted into the patient 460's body. When an infusion container 456 is initially at a height 404, increasing the height of the infusion container 456 to a height 406 increases a head height for the patient 460. For example, a smaller head height 452 is increased to a larger head height 454. Conversely, when the infusion container 456 is initially at the height 406, by lowering the infusion container 456 to the height 404, the head height for the patient 460 is reduced. For example, the larger head height 454 is reduced to a smaller head height 452. In some implementations, as shown in FIG. 4A, the infusion is a gravity infusion (e.g., gravity drip infusion), where no infusion pump is used in the infusion process, and the infusion container 456 is directly fluidically connected to the patient 460. Changing a height of the infusion container 456 changes the head height, which in turn changes an infusion flow rate to the patient 460. In some implementations, the head height is measured from a top fluid level in the infusion container 456 to the patient 460's heart. As the infusion process progresses, the volume of fluid in the infusion container 456 decreases, the top fluid level in the infusion container 456 falls, and the head height is reduced. To maintain a desired flow rate during the infusion process, the height of the infusion container 456 can be increased continuously (e.g., by the system 300 or the system 350) to maintain the appropriate head height to obtain the desired flow rate.

FIG. 4B shows a patient receiving an infusion fluid from an infusion container delivered by an infusion pump, according to some aspects of the subject technology. Changing a relative height between an infusion container 456 and the infusion pump 458 changes the hydrostatic pressure of the fluid from the infusion container 456 at infusion pump 458. For example, when the infusion container 456 is initially at a height 412, increasing the height of the infusion container 456 to a height 414 increases the hydrostatic pressure of the fluid from the infusion container 456 at the infusion pump 458. At the same time, the relative height difference between the infusion container 456 and the patient 460 also changes. In some implementations, the infusion pump 458 may counteract or further increase the head height between the infusion container 456 and the patient 460. Conversely, when the infusion container 456 is initially at the height 414, lowering the infusion container 456 to a height 412 decreases the hydrostatic pressure of the fluid from the infusion container 456 at the infusion pump 458. The height of the infusion container 456 can be varied using the system 300 or the system 350. In some implementations, the system 300 further includes an encoder (or another detector) to track current heights of the first arm 304 and the second arm 306, and the system 300 either senses a current height of the infusion pump 458 or receives the information from the infusion pump 458. Alternatively, in some implementations, the infusion pump 458 receives information about current heights of the first arm 304 and the second arm 306 from the system 300. Similarly, in some implementations, the system 350 may include an encoder or another detector to track a current height of the upper extension 354. The information about the current height of the upper extension 354 may be communicated to the infusion pump 458, or the current height of the infusion pump 458 may be communicated to the system 350.

A processor in or communicatively connected to one or more of the infusion pump 458, the system 300, or the system 350 determines a relative height difference between the infusion container 456 and the infusion pump 458. In some implementations, a calibration process determines how differences in hydrostatic pressure (caused by differences in relative heights of the infusion container 456 and the infusion pump 458) affect a flow rate and/or a fluidic pressure of the fluid delivered to the patient 460. For example, when the infusion container 456 is at the height 412, the hydrostatic pressure of the fluid at inlet of the infusion pump 458 is lower than the hydrostatic pressure when the infusion container 456 is at the height 414. The decrease in hydrostatic pressure may reduce a flow rate or a fluidic pressure of the fluid being received by the infusion pump 458. In some implementations, by varying the height of the infusion container 456, fine adjustments of the flow rate or fluid pressure of the fluid delivered to the patient 460 may be varied.

FIG. 4C shows an example in which a patient's position is adjusted, according to aspects of the subject technology. The patient 460 is initially situated at a height 422, as shown in FIG. 4C. Instead of a patient lying horizontally (e.g., in a hospital bed 462), the patient 460 may also be sitting or standing, and the height 422 may then be associated to a height of where the IV administration set is inserted into the patient 460's body. Both the infusion container 456, and the infusion pump 458 used to infuse the infusion container 456 are held stationary. The height of the patient 460 can be raised or lowered by a manually actuated or electrically actuated bed or chair. Raising the height of the patient 460 to a height 424 decreases a height difference between the patient 460 and the infusion pump 458. Reducing the height differential causes a hydrostatic pressure of the infusion fluid at the patient 460 to be lowered, when the infusion parameters of the infusion pump 458 are held constant. Conversely, when the patient 460 is initially at the height 424, by lowering the patient 460 to the height 422, a height difference between the patient 460 and the infusion pump 458 is increased.

Increasing the height differential causes a hydrostatic pressure of the infusion fluid at the patient 460 to be increased, when the infusion parameters of the infusion pump 458 are held constant. In some implementations, a calibration process determines how differences in hydrostatic pressure (caused by differences in relative heights of the infusion pump 458 and the patient 460) affect a flow rate and/or a fluidic pressure of the fluid delivered to the patient 460. For example, when the patient 460 is at the height 424, the hydrostatic pressure of the fluid at the location of delivery to the patient 460's body is lower than the hydrostatic pressure when the patient 460 is at the height 422. The decrease in hydrostatic pressure may reduce a flow rate or a fluidic pressure of the fluid being received by the patient 460.

In some implementations, by varying the height of the patient 460, fine adjustments of the flow rate or fluid pressure of the fluid delivered to the patient 460 may be varied. In some implementations, a height of the patient 460 may be continuously lowered during the infusion process, to counteract any decrease in head height (from the falling fluid level in the infusion container 456) while keeping infusion parameters of the infusion pump 458 constant. In some implementations, the bed 462 (or an adjustable chair) may include an encoder or another detector to track a current height of the bed 462. The information about the current height of the bed 462 may be communicated to the infusion pump 458. A processor in or communicatively connected to the infusion pump 458 determines a relative height difference between the infusion pump 458 and the bed 462, and causes the infusion pump 458 to send control signals to the bed 462 to vary a height of the 462 during the infusion process.

FIG. 4D shows an example in which a position of an infusion pump is changed, according to aspect of the subject technology. The patient 460 is maintained at a constant height 402, and the infusion container 456 is also held stationary. When the infusion pump 458, initially at a height 434, is raised to a height 436, a hydrostatic pressure of the infusion fluid at the inlet of the infusion pump 458 decreases. At the same time, a hydrostatic pressure of the fluid at an outlet of the infusion pump 458 is increased relative to the patient 460 positioned at the height 432. Conversely, when the infusion pump 458, initially at the height 436, is lowered to the height 434, a hydrostatic pressure of the infusion fluid at the inlet of the infusion pump 458 increases. At the same time, a hydrostatic pressure of the fluid at an outlet of the infusion pump 458 is decreased relative to the patient 460 positioned at the height 432. In other words, when the infusion pump 458 is at the height 434, the fluid from the infusion container 456 traverses a longer flow path, and experiences a greater height drop from the infusion container 456 to the infusion pump 458 and a shorter flow distance (and a smaller height drop) from the infusion pump 458 to the patient 460. In contrast, when the infusion pump 458 is at the height 436, the fluid traverses a shorter distance over a smaller height drop from the infusion container 456 to the infusion pump 458, and a longer distance (and a greater height drop) from the infusion pump 458 to the patients 460.

The combined effect of an increase in hydrostatic pressure at the inlet of the infusion pump 458 and a decrease in hydrostatic pressure at the outlet of the infusion pump 458 as a result of lowering the infusion pump 458 to the height 434 may result in either an increase or decrease in the flow rate and/or fluid pressure of the infusion fluid supplied to the patient 460. For example, characteristics of the infusion fluid (e.g., viscosity, or density, or volume), and the infusion process (e.g., flow rate, or VBTI) may impact the overall flow rate and fluid pressure delivered to the patient 460.

In some implementations, the infusion pump 458 may include a pressure sensor at or near an outlet of the infusion pump 458. Having the infusion pump 458 be closer in height to the patient 460 may increase a certainty of a fluid flow rate and fluid pressure that is being delivered to the patient 460. For example, characteristics of the flow would be measured by the pressure sensor at the outlet of the infusion pump 458 before too many variations (e.g., height drops) occur prior to the fluid being delivered to the patient 460. Such an arrangement might be suitable for an infusion process that requires greater precision.

In some implementations, instead of using a single infusion container 456, the secondary infusion container 310 and the primary infusion container 308 shown in FIG. 3A may be used in any of the arrangements shown in FIGS. 4A to 4D. For example, in FIG. 4D, lowering the infusion pump 458 to the height 434 effectively provides more space for the secondary infusion container 310 and the primary infusion container 308 to be separated by a greater head-height differential 334, reducing the likelihood of a concurrent flow.

In some implementations, the infusion pump 458 includes closed loop infusion control capabilities and modifies one or more operation parameters of the infusion pump based on the signal received from the system 300 or the system 350.

In some implementations, the processor of the system 300 or the system 350 may generate flow rate information based on detected weights or volumes over time. This flow rate information may be provided to the infusion pump 458 or compared, by the system 300 or system 350, with desired flow rate information received from the infusion pump 458. If the generated rate does not correspond to a desired flow rate, the adjustment device may adjust one or more physical element associated therewith. The adjustment may include activating a perceivable indicator, adjusting a display or other user interface, adjusting power supply or mode, stopping or slowing a motor, or the like.

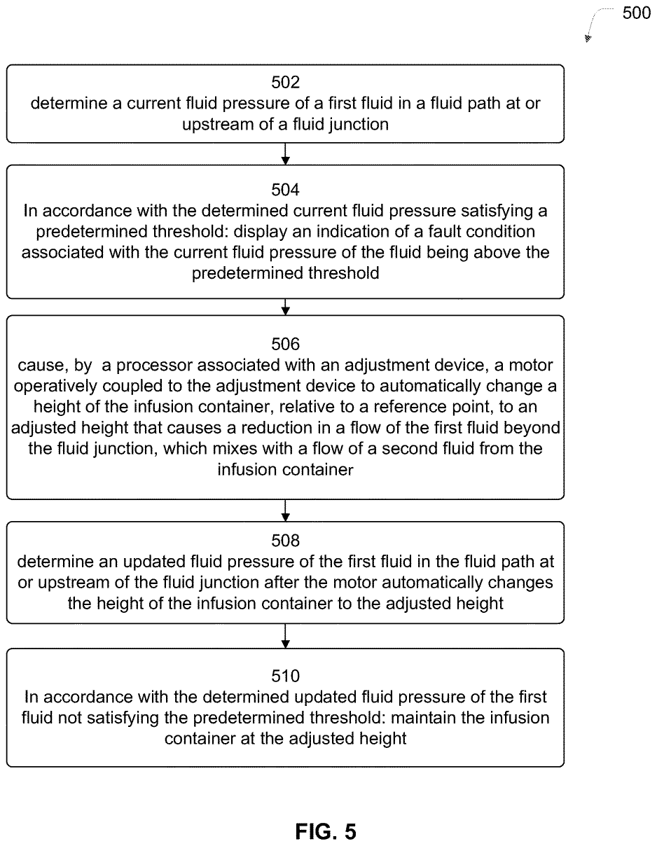

FIG. 5 depicts an example method for adjusting a position of an infusion container, according to aspects of the subject technology. For explanatory purposes, the various blocks of example method 500 may be described with reference to FIGS. 1-4D, and the components and/or processes described herein. The one or more of the blocks of method 500 may be implemented, for example, by one or more computing devices such as those described above. In some implementations, one or more of the blocks may be implemented apart from other blocks, and by one or more different processors or devices described. Further for explanatory purposes, the blocks of example method 500 are described as occurring in serial, or linearly. However, multiple blocks of example method 500 may occur in parallel. In addition, the blocks of example method 500 need not be performed in the order shown and/or one or more of the blocks of example method 500 need not be performed.

In the depicted example, during an ongoing infusion, a system determines a current fluid pressure of a first fluid in a fluid path at or upstream of a fluid junction (502). Determining the current fluid pressure of the first fluid may mean receiving an indication of the current fluid pressure of the first fluid. Detecting a current fluid pressure of a first fluid may be performed by pump sensors. Detecting a current fluid pressure and determining a current fluid pressure may be used interchangeably. In some implementations, the fluid pressure may be determined during an administration of the first fluid to a patient. In some implementations, the first fluid may be gravity administered. The current fluid pressure may be detected using a pressure sensor of an infusion pump that detects a fluid pressure upstream of the pump (e.g., measuring a joint fluid pressure of a flow from a secondary infusion container and a flow from a primary infusion container) or using a pressure sensor provided near (e.g., integrated within, upstream of, or downstream of) a backcheck valve or detected using a pressure sensor provided near a drip chamber. In some implementations, an infusion device 208 may be administering the first fluid to the patient. In accordance with the determined current fluid pressure satisfying a predetermined threshold: display an indication of a fault condition associated with the current fluid pressure of the fluid being above the predetermined threshold: the system displays an indication of a fault condition associated with the current fluid pressure of the fluid being above the predetermined threshold (504).

The system causes, via a processor associated with an adjustment device, a motor operatively coupled to the adjustment device to automatically change a height of the infusion container, relative to a reference point, to an adjusted height that causes a reduction in a flow of the first fluid beyond the fluid junction, which mixes with a flow of a second fluid from the infusion container (506). The system determines an updated fluid pressure of the first fluid in the fluid path at or upstream of the fluid junction after the motor automatically changes the height of the infusion container to the adjusted height (508). In accordance with the determined updated fluid pressure of the first fluid in the fluid path at or upstream of the fluid junction not satisfying the predetermined threshold: the system maintains the infusion container at the adjusted height (510).

The system 300 and the system 350 may be used with infusion pumps and provide some of the guardrails, monitoring, alerting, and connectivity features. For example, the system 300 and the system 350 can be used with a pump specifically configured to provide fluid pressure, mass, and/or flow rate information to the system 300 and/or the system 350 either by direct communication with the pump, or via interoperability with a hospital network. The system 300 and the system 350 can also be used alone for gravity infusions to provide certain features described. The Bluetooth communication circuitry inside the system 300 and the system 350 can also provide various feedback to a user (e.g., care provider or the patient) or receive input from a user with a tablet or mobile phone application.

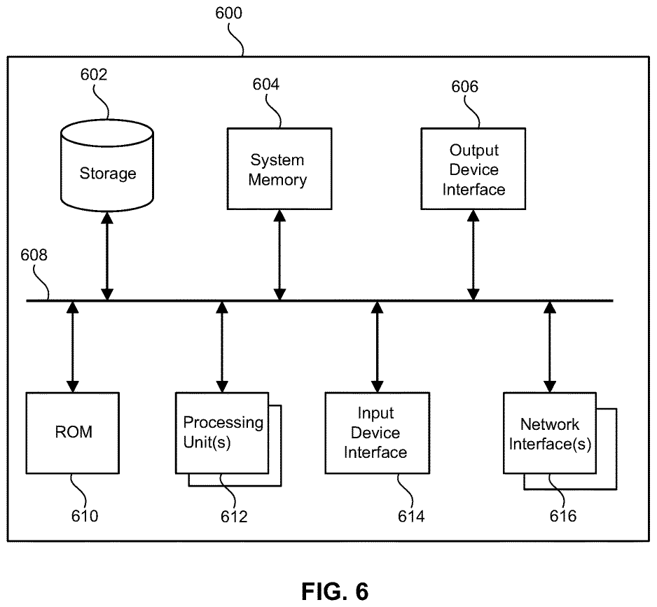

FIG. 6 is a conceptual diagram illustrating an example electronic system for determining a fault condition during an infusion process, according to aspects of the subject technology. Electronic system 600 may be a specifically configured computing device for execution of software associated with one or more portions or steps of method 500, or components and processes provided by FIGS. 1-5, including but not limited to server 130, computing hardware within patient care device 12, or terminal device 132. Electronic system 600 may be representative, in combination with the disclosure regarding FIGS. 1-5. In this regard, electronic system 600 may be a specifically configured personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or other sort of computer-related electronic device having network connectivity.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and processes previously described.

Bus 608 collectively represents system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves instructions to execute and data to process in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such a random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry (e.g., transceiver, antenna, amplifier) for connecting to a wireless access point. Network interfaces 616 may also include hardware (e.g., Ethernet hardware) for connecting the computer to a part of a network of computers such as a local area network (“LAN”), a wide area network (“WAN”), wireless LAN, personal area network (“PAN”), or an Intranet, or a network of networks, such as the Internet. Any or all components of electronic system 600 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices specifically configured for the infusion features described can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a specifically configured computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”), a personal area network (“PAN”), a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

In some implementations, a method of adjusting a position of an infusion container includes determining a current fluid pressure of a first fluid in a fluid path at or upstream of a fluid junction. In accordance with the determined current fluid pressure satisfying a predetermined threshold: displaying an indication of a fault condition associated with the current fluid pressure of the fluid being above the predetermined threshold; and causing, by a processor associated with an adjustment device, a motor operatively coupled to the adjustment device to automatically change a height of the infusion container, relative to a reference point, to an adjusted height that causes a reduction in a flow of the first fluid beyond the fluid junction, which mixes with a flow of a second fluid from the infusion container; and determining an updated fluid pressure of the first fluid in the fluid path at or upstream of the fluid junction after the motor automatically changes the height of the infusion container to the adjusted height; in accordance with the determined updated fluid pressure of the first fluid not satisfying the predetermined threshold: maintaining the infusion container at the adjusted height.

For example, in some implementations, the method of adjusting the position of the infusion container includes adjusting a head height associated with the infusion container.

In some implementations, the current fluid pressure of the first fluid in the fluid path is determined upstream of a check valve (e.g., a backcheck valve). In some gravity infusion processes, the determination is an indirect detection that infers a pressure of the first fluid in the fluid path by detecting the formation of drops in a drip chamber upstream of the check valve. In some implementations, the current fluid pressure is determined using a pressure sensor of the infusion pump that detects a fluid pressure upstream of the pump (e.g., measuring a joint fluid pressure of a flow from the secondary infusion container and a flow from the primary infusion container). In some implementations, the current fluid pressure is determined using a pressure sensor provided near (e.g., integrated within, upstream of, or downstream of) a backcheck valve or determined using a pressure sensor provided near the drip chamber.

In some implementations, the check valve is configured to stop the first fluid from flowing beyond the check valve. In some implementations, displaying an indication of the fault condition includes displaying an indication of the fault condition on a display of the infusion device, or a display of a separate user device (e.g., a phone, or a tablet) to provide an alert to the user. In some implementations, automatically changing a height of the infusion container includes using a pivot that adjusts a first height of a first arm extending from the pivot and a second height of a second arm extending from the pivot, or adjusting a height of an infusion container. In some implementations, adjusting a head height associated with the infusion container further includes adjusting a height of a patient's bed, adjusting a height of a patient's chair, and/or adjusting a height of an infusion pump. In some implementations, adjusting a position of the infusion container includes adjusting a height of a primary container independently of a height of the infusion container (e.g., a secondary container).

In some implementations, determining the current fluid pressure of the first fluid in the fluid path at or upstream of the fluid junction includes determining a pressure differential across a check valve positioned at the fluid junction. The check valve is configured to stop the first fluid from flowing beyond the fluid junction during the flow of the second fluid from the infusion container; and wherein an increase in the pressure differential across the check valve causes the first fluid to flow beyond the fluid junction.

In some implementations, the infusion container is a secondary infusion container, the method further includes monitoring (e.g., using the second weight sensor that senses a decrease in weight in the primary infusion container, by visual sensing a level of fluid in the primary infusion container, or using a pressure sensor to detect a pressure spike in a fluid path of the first fluid in the primary infusion container) a sympathetic flow between the second fluid in the secondary infusion container and the first fluid in a primary infusion container. The processor causes an output of an infusion device to indicate the fault condition when the sympathetic flow causes a mixing of more than a threshold percentage of the first fluid from the primary infusion container (e.g., the primary infusion container is connected to a second weight sensor) into a flow of the second fluid from the secondary infusion container.

In some implementations, the reference point is a position of the primary infusion container, and in response to determining that the current fluid pressure of the first fluid in the fluid path at or upstream of the fluid junction is above the predetermined threshold, causing the motor operatively coupled to the adjustment device to automatically adjust a height of the secondary infusion container relative to the height of the primary infusion container by changing the height of the secondary infusion container independently of a height of the primary infusion container to change a head-height differential between the primary infusion container and the second infusion container so that the sympathetic flow is reduced.

In some implementations, adjusting the height of the secondary infusion container includes pivoting, by the motor, a first arm of a structure holding the secondary infusion container relative to the height of the primary infusion container.

In some implementations, the method further includes determining a modified height of the secondary infusion container relative to the primary infusion container based on an upstream pressure, a programmed infusion rate, and a volume to be infused; and causing the motor operatively coupled to the adjustment device to automatically adjust a height of the secondary infusion container and/or a height of the primary infusion container so that the primary infusion container and the secondary infusion container is separated by the modified height.

In some implementations, displaying the indication of the fault condition includes presenting on a display of the infusion device or a separate user device an alert to a user to change a height difference between the primary infusion container and the secondary infusion container and/or an indication that the motor commences to automatically adjust the height difference.

In some implementations, the reference point is a height of a patient, the method further includes: responsive to determining that a fluid pressure between the infusion container and the patient fails to satisfy a first threshold, adjusting, by the motor operatively coupled to the adjustment device a height of the infusion container relative to the height of the patient by changing the height of the patient so that a fluid pressure between the infusion container and the patient is above the first threshold. In some implementations, satisfying the first threshold includes meeting and/or being higher than the first threshold.

In some implementations, the reference point is a height of a patient, the method further comprising: responsive to determining that a fluid pressure between the infusion container and the patient fails to satisfy a first threshold, adjusting, by the motor operatively coupled to the adjustment device a height of the infusion container relative to the height of the patient by changing a height of the infusion container so that a fluid pressure between the infusion container and the patient satisfies the first threshold.

In some implementations, the reference point is a height of an infusion pump, and the method includes automatically adjusting, via the motor operatively coupled to the adjustment device, a height of the infusion container relative to the height of the infusion pump by changing the height of the infusion pump.

In some implementations, the method further includes: responsive to determining that a pressure between the infusion pump and a patient is lower than a first threshold, driving, by the motor, a portion of a telescopic pole coupled to the infusion pump to increase a height of the infusion pump relative to the patient so that a pressure of the fluid delivered to the patient is increased.

In some implementations, the reference point is a height of an infusion pump, the method further comprising: responsive to determining that a fluid pressure between the infusion container and the infusion pump is lower than a first threshold, adjusting, by the motor operatively coupled to the adjustment device a height of the infusion container relative to the height of the infusion pump by changing the height of the infusion container so that a fluid pressure between the infusion container and the infusion pump is above the first threshold.

In some implementations, motor is configured to drive a portion of a telescopic pole coupled to the infusion container to increase a height of the infusion container relative to the infusion pump so that a pressure of the fluid delivered to the infusion pump is increased by increasing a separation between telescopic members of the telescopic pole.

In some implementations, the height of the infusion container relative to the reference point is adjusted at a plurality of time points using a closed loop control system.

In some implementations, the method includes receiving, as input to the closed loop control system, a current characteristic of the infusion container, a height of one or more: the infusion container, the infusion pump, and a patient fluidically connected to the infusion container; properties of an administration set, and set parameters of an infusion process; and adjusting, via the motor operatively coupled to the adjust device, a height of one or more of: the infusion container, the infusion pump, or the patient based on an output of the closed loop control system to reduce a deviation of current infusion characteristics from the set parameters.

In some implementations, receiving the current characteristic of the infusion container includes receiving a current mass of the infusion container and a height of a fluid in the infusion container. In some implementations, receiving a current characteristic of the infusion container includes receiving a height of the fluid in the infusion container using image recognition techniques on an image to analyze one or more visual markers that are indicative of the height of the fluid in the infusion container.

In some implementations, the method further includes monitoring, via a vision system, a fluid level of the first fluid and or a presence of drops of first fluid in a drip chamber connected to an infusion container of the first fluid.

In some implementations, the method further includes determining (e.g., by receiving a programmed flow rate pertaining the fluid infusion; programmed flow rate sent from a central medical network; flow rate entered locally by a practitioner at the infusion device, scanned by the practitioner at the infusion device) a desired flow rate for the fluid in the infusion container (e.g., the infusion container is a secondary container); determining a current flow rate (e.g., by receiving a measurement of a mass of the infusion container) of the fluid in the infusion container and responsive to a determination that a difference between the current flow rate and the desired flow rate exceeds a second threshold, causing the motor to change a height of the infusion container to reduce a magnitude of a difference between the current flow rate and the desired flow rate.

In some implementations, determining the current flow rate includes determining, by the processor, at a first time, using a weight sensor communicably connected to the processor and an infusion device, a first weight of an infusion container associated with the fluid infusion provided by the infusion device; determining, by the processor, at a second time, using the weight sensor, a second weight of the infusion container; determining, by the processor, based on the first weight and the second weight, the determined flow rate of the fluid infusion provided by the infusion device between the first time and the second time.

In some implementations, determining the desired flow rate includes receiving, by a processor, from an infusion device, a programmed flow rate pertaining to a fluid infusion provided by the infusion device.

In some implementations, the method further includes continuing to automatically change the height of the infusion container until the current fluid pressure of the first fluid in the fluid path at or upstream of the fluid junction is below the predetermined threshold.

In some implementations, the method further includes continually monitoring a pressure of the first fluid as the second fluid from the infusion container is depleted during an infusion process; and adjusting a height of the infusion container based on the pressure of the first fluid.

In some embodiments, an infusion system includes a pressure sensor configured to detect a current fluid pressure of a first fluid in a fluid path at or upstream of a fluid junction; a processor, configured to receive information about a predetermined threshold of a fluid pressure; a motor, operatively coupled to an adjustment device, to automatically adjust a height of an infusion container relative to a reference point to an adjusted height in response to a determination that the current fluid pressure of the first fluid in the fluid path at or upstream of the fluid junction satisfies the predetermined threshold of the fluid pressure, that causes a reduction in a flow of the first fluid beyond the fluid junction, which mixes with a flow of a second fluid from the infusion container; and a display configured to present an indication of a fault condition associated with the current fluid pressure of the fluid satisfying the predetermined threshold.

In some implementations, the infusion system further includes an infusion pump configured to receive the second fluid from the infusion container and to deliver the second fluid in the infusion container to a patient.

In some implementations, the infusion system further includes a pivot connected to a first arm and a second arm of a structure. The first arm of the structure is configured to hold the infusion container and a second arm of the structure is configured to hold a second infusion container, and the motor is configured to automatically adjust a relative height between the first arm and the second arm of the structure based on the current fluid pressure of the first fluid.

In some implementations, a non-transitory computer-readable storage medium, storing a computer program, the computer program, when executed by one or more processors of an electronic device, cause the one or more processors to perform operations including: determining a current fluid pressure of a first fluid in a fluid path at or upstream of a fluid junction. In accordance with the determined current fluid pressure satisfying a predetermined threshold: displaying an indication of a fault condition associated with the current fluid pressure of the fluid being above the predetermined threshold; and causing, by a processor associated with an adjustment device, a motor operatively coupled to the adjustment device to automatically change a height of the infusion container, relative to a reference point, to an adjusted height that causes a reduction in a flow of the first fluid beyond the fluid junction, which mixes with a flow of a second fluid from the infusion container; and determining an updated fluid pressure of the first fluid in the fluid path at or upstream of the fluid junction after the motor automatically changes the height of the infusion container to the adjusted height; in accordance with the determined updated fluid pressure of the first fluid not satisfying the predetermined threshold: maintaining the infusion container at the adjusted height.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The term website, as used herein, may include any aspect of a website, including one or more web pages, one or more servers used to host or store web related content, etc. Accordingly, the term website may be used interchangeably with the terms web page and server. The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all implementations, or one or more implementations. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some embodiments, include a signal utilized to transmit one or more representations of the information. While recited in the singular, it will be understood that a message may be composed, transmitted, stored, received, etc. in multiple parts.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or cellular networks.

1. A method of adjusting a position of an infusion container, the method comprising, under control of at least one electronic device:
determining a current fluid pressure of a first fluid in a fluid path at or upstream of a fluid junction;
in accordance with the determined current fluid pressure satisfying a predetermined threshold:
displaying an indication of a fault condition associated with the current fluid pressure being above the predetermined threshold; and
causing, via a processor associated with an adjustment device, a motor operatively coupled to the adjustment device to automatically change a height of the infusion container, relative to a reference point, to an adjusted height that causes a reduction in a flow of the first fluid beyond the fluid junction, which mixes with a flow of a second fluid from the infusion container;
determining an updated fluid pressure of the first fluid in the fluid path at or upstream of the fluid junction after the motor automatically changes the height of the infusion container to the adjusted height; and
in accordance with the determined updated fluid pressure of the first fluid not satisfying the predetermined threshold: maintaining the infusion container at the adjusted height.

2. The method of claim 1, wherein determining the current fluid pressure of the first fluid in the fluid path at or upstream of the fluid junction comprises determining a pressure differential across a check valve positioned at the fluid junction, wherein the check valve is configured to stop the first fluid from flowing beyond the fluid junction during the flow of the second fluid from the infusion container; and wherein an increase in the pressure differential across the check valve causes the first fluid to flow beyond the fluid junction.

3. The method of claim 1, wherein the infusion container is a secondary infusion container, the method further comprising:
monitoring a sympathetic flow between the second fluid in the secondary infusion container and the first fluid in a primary infusion container; and
causing, by the processor, an output of an infusion device to indicate the fault condition when the sympathetic flow causes a mixing of more than a threshold percentage of the first fluid from the primary infusion container into a flow of the second fluid from the secondary infusion container.

4. The method of claim 3, wherein the reference point is a position of the primary infusion container, the method further comprising:
in response to determining that the current fluid pressure of the first fluid in the fluid path at or upstream of the fluid junction is above the predetermined threshold, causing the motor operatively coupled to the adjustment device to automatically adjust a height of the secondary infusion container relative to the height of the primary infusion container by changing the height of the secondary infusion container independently of a height of the primary infusion container to change a head-height differential between the primary infusion container and the second infusion container such that the sympathetic flow is reduced.

5. The method of claim 4, wherein adjusting the height of the secondary infusion container comprises pivoting, by the motor, a first arm of a structure holding the secondary infusion container relative to the height of the primary infusion container.

6. The method of claim 3, further comprising:
determining a modified height of the secondary infusion container relative to the primary infusion container based on an upstream pressure, a programmed infusion rate, and a volume to be infused; and
causing the motor operatively coupled to the adjustment device to automatically adjust a height of the secondary infusion container and/or a height of the primary infusion container so that the primary infusion container and the secondary infusion container are separated by the modified height.

7. The method of claim 3, wherein displaying the indication of the fault condition comprises presenting on a display of the infusion device or a separate user device an indication that the motor commences to automatically adjust the height difference.

8. The method of claim 1, wherein the reference point is a height of a patient, the method further comprising:
responsive to determining that a fluid pressure between the infusion container and the patient is lower than a first threshold, adjusting, by the motor operatively coupled to the adjustment device a height of the infusion container relative to the height of the patient by changing the height of the patient so that a fluid pressure between the infusion container and the patient is above the first threshold.

9. The method of claim 1, wherein the reference point is a height of a patient, the method further comprising:
responsive to determining that a fluid pressure between the infusion container and the patient fails to satisfy a first threshold, adjusting, by the motor operatively coupled to the adjustment device a height of the infusion container relative to the height of the patient by changing a height of the infusion container so that a fluid pressure between the infusion container and the patient satisfies the first threshold.

10. The method of claim 1, wherein the reference point is a height of an infusion pump, the method further comprising:
automatically adjusting, via the motor operatively coupled to the adjustment device, a height of the infusion container relative to the height of the infusion pump by changing the height of the infusion pump.

11. The method of claim 10, further comprising:
responsive to determining that a pressure between the infusion pump and a patient is lower than a first threshold, driving, by the motor, a portion of a telescopic pole coupled to the infusion pump to increase a height of the infusion pump relative to the patient so that a pressure of the fluid delivered to the patient is increased.

12. The method of claim 1, wherein the reference point is a height of an infusion pump, the method further comprising:
responsive to determining that a fluid pressure between the infusion container and the infusion pump is lower than a first threshold, adjusting, by the motor operatively coupled to the adjustment device a height of the infusion container relative to the height of the infusion pump by changing the height of the infusion container so that a fluid pressure between the infusion container and the infusion pump is above the first threshold.

13. The method of claim 12, further comprising driving the motor a portion of a telescopic pole coupled to the infusion container to increase a height of the infusion container relative to the infusion pump so that a pressure of the fluid delivered to the infusion pump is increased by increasing a separation between telescopic members of the telescopic pole.

14. The method of claim 1, wherein the height of the infusion container relative to the reference point is adjusted at a plurality of time points using a closed loop control system.

15. The method of claim 14, the method further comprising:
receiving, as input to the closed loop control system, a current characteristic of the infusion container, a height of one or more: the infusion container, the infusion pump, and a patient fluidically connected to the infusion container; properties of an administration set, and set parameters of an infusion process; and
adjusting, via the motor operatively coupled to the adjustment device, a height of one or more of: the infusion container, the infusion pump, or the patient based on an output of the closed loop control system to reduce a deviation of current infusion characteristics from the set parameters.

16. The method of claim 15, wherein receiving the current characteristic of the infusion container comprises receiving a current mass of the infusion container and a height of a fluid in the infusion container.

17. The method of claim 1, further comprising:
monitoring, via a vision system, a fluid level of the first fluid and/or a presence of drops of first fluid in a drip chamber connected to an infusion container of the first fluid.

18. The method of claim 1, further comprising determining a desired flow rate for the fluid in the infusion container; determining a current flow rate of the fluid in the infusion container and responsive to a determination that a difference between the current flow rate and the desired flow rate exceeds a second threshold, causing the motor to change a height of the infusion container to reduce a magnitude of a difference between the current flow rate and the desired flow rate.

19. The method of claim 18, wherein determining the current flow rate comprises:
determining, by the processor, at a first time, using a weight sensor communicably connected to the processor and an infusion device, a first weight of an infusion container associated with a fluid infusion provided by the infusion device;
determining, by the processor, at a second time, using the weight sensor, a second weight of the infusion container; and
determining, by the processor, based on the first weight and the second weight, the determined flow rate of the fluid between the first time and the second time.

20. The method of claim 19, wherein determining the desired flow rate comprises receiving, by a processor, from an infusion device, a programmed flow rate pertaining to a fluid infusion provided by the infusion device.

21. The method of claim 2, further comprising continuing to automatically change the height of the infusion container until the current fluid pressure of the first fluid in the fluid path at or upstream of the fluid junction is below the predetermined threshold.

22. The method of claim 1, further comprising continually monitoring a pressure of the first fluid as the second fluid from the infusion container is depleted during an infusion process; and adjusting a height of the infusion container based on the pressure of the first fluid.

23. An infusion system, comprising:
a pressure sensor configured to detect a current fluid pressure of a first fluid in a fluid path at or upstream of a fluid junction;
a processor, configured to receive information about a predetermined threshold of a fluid pressure;
a motor, operatively coupled to the processor, to automatically adjust a height of an infusion container relative to a reference point to an adjusted height in response to a determination that the current fluid pressure of the first fluid in the fluid path at or upstream of the fluid junction satisfies the predetermined threshold of the fluid pressure, that causes a reduction in a flow of the first fluid beyond the fluid junction, which mixes with a flow of a second fluid from the infusion container; and
a display configured to present an indication of a fault condition associated with the current fluid pressure of the fluid satisfying the predetermined threshold.

24. The infusion system of claim 23, further comprising, an infusion pump configured to receive the second fluid from the infusion container and to deliver the second fluid in the infusion container to a patient.

25. The infusion system of claim 23, further comprising a pivot connected to a first arm and a second arm of a structure, wherein the first arm of the structure is configured to hold the infusion container and a second arm of the structure is configured to hold a second infusion container, and the motor is configured to automatically adjust a relative height between the first arm and the second arm of the structure based on the current fluid pressure of the first fluid.

26. A non-transitory computer-readable storage medium, storing a computer program, the computer program, when executed by one or more processors of an electronic device, cause the one or more processors to perform operations comprising:
determining a current fluid pressure of a first fluid in a fluid path at or upstream of a fluid junction;
in accordance with the determined current fluid pressure satisfying a predetermined threshold:
displaying an indication of a fault condition associated with the current fluid pressure being above the predetermined threshold; and
causing, by a processor associated with an adjustment device, a motor operatively coupled to the adjustment device to automatically change a height of an infusion container, relative to a reference point, to an adjusted height that causes a reduction in a flow of the first fluid beyond the fluid junction, which mixes with a flow of a second fluid from the infusion container;
determining an updated fluid pressure of the first fluid in the fluid path at or upstream of the fluid junction after the motor automatically changes the height of the infusion container to the adjusted height; and
in accordance with the determined updated fluid pressure of the first fluid not satisfying the predetermined threshold: maintaining the infusion container at the adjusted height.
